(19) United States
(12) Patent Application Publication
(10) Pub. No.: US 2006/0058263 A1
(43) Pub. Date: Mar. 16, 2006

(54) Heat pasturized liquids containing glucosamine

(76) Inventors: Brent Daniel Rogers, Ottumwa, IA (US); Lawrence E. Fosdick, Oskallosa, IA (US); John A. Bohlmann, Ottumwa, IA (US)

Correspondence Address: CARGILL, INCORPORATED, LAW DEPARTMENT, P. O. BOX 5624, MINNEAPOLIS, MN, 55440-5624, US

(21) Appl. No.: 10/533,412

(22) PCT Filed: Oct. 31, 2003
(86) PCT No.: PCT/US03/34844

§ 371 Date: Apr. 29, 2005

Related U.S. Application Data
(60) Provisional application No. 60/423,119, filed on Nov. 1, 2002.

(30) Foreign Application Priority Data
Dec. 19, 2002 (US) 10/326549
Oct. 13, 2003 (US) 10/685125

(51) Int. Cl. A61K 31/7008

(52) U.S. Cl. 514/62

(57) ABSTRACT

The disclosure provides methods of making heat-pasteurized liquids, such as beverages, that contain glucosamine, wherein glucosamine is present in the beverage during the pasteurization process. The disclosure also provides liquids, such as beverages, made by these methods, as well as methods of using the glucosamine supplemented liquids, for example to treat osteoarthritis.

### CROSS REFERENCE TO RELATED APPLICATIONS

This application claims the benefit of U.S. Provisional Application No. 60/423,119, filed Nov. 1, 2002, and is a continuation-in-part of U.S. patent application Ser. No. 10/685,125 filed Oct. 13, 2003, which is a continuation-in-part of copending U.S. patent application Ser. No. 10/326,549, filed Dec. 19, 2002, which is a continuation of U.S. patent application Ser. No. 09/785,695, filed Feb. 16, 2001, and which claims priority from PCT Application No. PCT/US02/04468, filed Feb. 15, 2002, each of which is incorporated herein by reference.

### FIELD

This application relates to heat pasteurized liquids, such as beverages, condiments, and soups that include glucosamine and methods of making and using such compositions.

### BACKGROUND

Beverage supplements can supply consumers with dietary supplements or the necessary vitamins and minerals specified in the recommended daily allowances (RDA) provided by the U.S. government. Examples of nutritionally-balanced beverages are disclosed in U.S. Pat. Nos. 3,894,148; 4,309,417; 4,312,856; 4,322,407; 6,432,929; and 6,391,864 as well as EP Application No. EP 0 681 434.

Dietary supplements for cartilage health and maintenance are effective in reducing the symptoms of osteoarthritis and,joint pain. Examples of cartilage supplements include glucosamine (GLCN) hydrochloride, GLCN sulfate, N-acetyl-D-glucosamine (NAG), chondroitin sulfate, hyaluronic acid (which is comprised of a repeating disaccharide of N-acetyl-D-glucosamine and D-glucuronic acid), and cetyl myristoleate (CM). The most commonly used cartilage supplements are GLCN hydrochloride and GLCN sulfate.

It has been disclosed and the industry has followed the belief that exposure of GLCN to relatively high temperatures used in food processing applications, such as pasteurization, would inactivate GLCN (for example, see U.S. Pat. No. 6,423,929). In attempt to overcome this limitation, U.S. Pat. No. 6,423,929 teaches that beverages that include GLCN are prepared using a process that requires two separate heating steps to minimize chemical alteration of GLCN. A juice drink base (without GLCN) is prepared using pasteurization at about 195° F. for 42 seconds. A separate GLCN water-based solution is prepared at a temperature of below 160° F., such that the GLCN is not inactivated. The juice drink base and the GLCN solution are then mixed to form a GLCN-supplemented beverage. Processing a beverage using two different solutions at two different temperatures could be relatively expensive and difficult to implement.

### SUMMARY

Disclosed are GLCN-containing liquids, such as beverages, soups, and condiments, which are treated under high-temperature conditions, such as those used in heat-pasteurization, without a significant amount of the GLCN being inactivated due to exposure to the high temperature. It has surprisingly been found that GLCN-supplemented liquids, such as beverages, can be made under high-temperature heating conditions that mimic pasteurization without substantially inactivating or degrading the GLCN. In addition, in certain embodiments, the GLCN-supplemented liquids, such as beverages, exposed to high temperatures do not have an unpleasant taste, off color, or off odor.

### DETAILED DESCRIPTION OF SEVERAL EMBODIMENTS

### Abbreviations and Terms

The following explanations of terms and methods are provided to better describe the present disclosure and to guide those of ordinary skill in the art in the practice of the present disclosure. As used herein and in the claims, the singular forms “a” or “an” or “the” include plural references unless the context clearly dictates otherwise. For example, reference to “a supplement” includes a plurality of such supplements and reference to “the beverage” includes reference to one or more beverages and equivalents thereof known to those skilled in the art, and so forth. Similarly, the word “or” is intended to include “and” unless the context clearly indicates otherwise. Hence “comprising A or B” means including A, or B, or A and B.

Unless otherwise indicated, all numbers expressing quantities of ingredients, temperatures, time periods, and so forth used in the specification and claims are to be understood as being modified by the term “about” whether explicitly stated or not. Accordingly, unless indicated clearly to the contrary, the numerical parameters set forth are approximations.

Unless explained otherwise, all technical and scientific terms used herein have the same meaning as commonly understood to one of ordinary skill in the art to which this disclosure belongs.

Administer: To cause a subject to receive something. As used herein, administration of the disclosed beverages supplemented with GLCN is oral, for example by ingestion.

Beverage: Any drink suitable for ingestion. Includes ready-to-drink beverages in their liquid form, such as juice or soda, concentrates, as well as those in a dry or powered form, such as a tea, instant coffee, or hot chocolate mix. Non-limiting examples of beverages that can be supplemented with GLCN include naturally or artificially flavored fruit or vegetable juices; milk; commercially available sports drinks (sugar or juice based) such as Gatorade®, Powerade®, and Allsport®; soda; Tang®; flavored waters; soy milk; and commercially available nutritionally-balanced beverages such as Ensure® beverage. A beverage can be carbonated or non-carbonated. Alcoholic beverages are also encompassed by this disclosure, such as wine, wine coolers, malt beverages and coolers, and beer.

Cartilage dysfunction: A disorder in a subject that results in joint pain or decreased joint mobility, for example arthritis, such as osteoarthritis.

Cartilage supplement: An agent that reduces joint pain, increases joint mobility, reduces swelling, or stimulates joint healing in a subject. In particular examples, it is an agent that delays or halts the onset of osteoartfritis. Examples include, but are not limited to: glucosamine, chondroitin sulfate, hyaluronic acid, chitin, cetyl myristoleate, essential fatty acids, MSM, SAMe, oligoglucosamine, and oligomers of N-acetyl-D-glucosamine (NAG).

Comprises: A term that means “including.”

Glucosamine (GLCN): As used herein, the term GLCN refers to the various forms of GLCN, such as agents having the general formula represented below, as well as salt complexes and substituted GLCN.

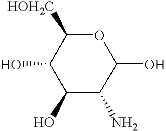

Salts of GLCN include, but are not limited to: citrate, acetate, phosphate, sulfate, chloride, lactate, and gluconate. Examples of GLCN derivatives include glucosamine itself, glucosamine hydrochloride, glucosamine hydroiodide, glucosamine chlorhydrate, glucosamine sulphate and N-acetyl glucosamine.

GLCN can be obtained from any suitable source. In certain examples, GLCN is a GLCN composition that is derived from shellfish, animal cartilage, bacteria, and/or fungal biomass.

In particular examples, GLCN is a GLCN composition that is derived from fungal biomass containing chitin. Suitable starting materials include microbial fungal sources, such as fungal sources derived from Aspergillus sp., Penicillium sp., Mucor sp., and combinations thereof. When GLCN is derived from fungal biomass, it will not pose a hazard to persons who have shellfish allergies because tropomyosin and other such muscle-derived proteins are not present in fungal biomass. Therefore, beverages containing GLCN-derived from fungal biomass will be tolerated by persons who have shellfish allergies. In addition, because GLCN derived from fungal biomass is not derived from shellfish (or any animal source), such GLCN-containing beverages are qualified for kosher status and may be consumed by strict vegetarians.

GLCN beverage: A beverage that contains GLCN, for example at least about 1 mg per serving, at least about 100 mg per serving, at least about 250 mg per serving, at least about 500 mg per serving, at least about 750 mg per serving, at least about 1 g per serving, or even at least about 20 g per serving. A pasteurized GLCN beverage is one that includes GLCN in the beverage when the beverage is exposed to high temperatures, such as those used in heat pasteurization.

High Temperature: As used herein, refers to temperatures typically used when liquids, such as beverages, are heat pasteurized, for example to destroy undesirable microorganisms.

Particular, non-limiting examples of high temperatures include, but are not limited to temperatures used in heat pasteurization, for example high temperatures are those at least about 160° F. (about 71° C.), such as temperatures of at least about 161° F. (about 71.5° C.), at least about 180° F. (82° C.), at least 194° F. (about 90° C.), at least about 200° F. (about 94° C.), at least about 212° F. (about 100° C.), at least about 220° F. (about 104° C.), at least about 280° F. (about 138° C.), or at least about 300° F. (about 149° C.). In particular examples, high temperatures include temperatures in the range of about 161° F. to about 300° F., such at about 161° F. to about 220° F., about 161° F. to about 212° F., about 161° F. to about 200° F., 165° F. to about 220° F., 165° F. to about 212° F., about 176° F. to about 220° F., or about 176° F. to about 212° F.

A GLCN-supplemented liquid, such as a GLCN beverage, can be exposed to a high temperature for an amount of time as needed to achieve a desired effect, for example to destroy objectionable microorganisms, such as to heat-pasteurize or to bring a GLCN liquid to a boil. Exemplary times include, but are not limited to, at least 15 seconds at a temperature of at least 160° F., at least 30 seconds at a temperature of at least 160° F., such as at least 20 minutes at about 160° F., at least 20 seconds at a temperature of about 194° F., at least 40 seconds at a temperature of about 194° F., or at least 60 seconds at 212° F.

Liquid: A substance in the fluid state of matter having little or no fixed shape but a fixed volume. Exemplary liquids include, but are not limited to, beverages, soups, yogurt, condiments such as ketchup and mustard, dressing, eggs, syrups, oils, gravies, pudding, and ice cream at a temperature above about 4° C.

Pasteurization: A method used to significantly reduce the presence of objectionable organisms (such as bacteria) in liquids (such as beverages) by exposing the liquid to heat, filtration, or irradiation for a period of time.

When a GLCN-supplemented liquid is exposed to a high temperature, the term “heat pasteurization” is used. In some embodiments, heat pasteurized liquids are subsequently cooled quickly to about 38° F. to 40° F. to retard the growth of surviving organisms. In particular embodiments, heat pasteurization does not substantially chemically alter a liquid, such as beverage or a supplement therein, and does not substantially affect the taste or mouthfeel of the liquid. As used herein, “heat pasteurization” or “heat pasteurized” do not include pasteurization by filtration, or irradiation.

In particular examples, heat pasteurization reduces the number of colony forming units (cfus) present in a liquid, such as a GLCN beverage, by at least 50%, such as at least 70%, at least 75%, at least 80%, at least 90%, at least 95%, or even at least 98%.

Particular examples of heat pasteurization temperatures are provided above in under “high temperature”. In some embodiments, heat pasteurization temperatures and incubation times include, but are not limited to: about 161° F. for about 15 seconds, about 195° F. for about 42 seconds (such as about 195±4° F. for about 42±4 seconds), about 200° F. for less than about 40 seconds (such as about 200±5° F. for about 40±5 seconds), about 165° F. for about 3 minutes (such as about 165±5° F. for about 180±10 seconds), and at least 280° F. for about 1-2 seconds (for example to ultrapasteurize milk).

If ultrapasteurization is desired, heat pasteurization temperatures can be increased to about 280° F. or greater (such as about 300° F.), with incubation for a shorter period of time, such as 1-2 seconds.

Preventing disease: A therapeutic intervention that inhibits the full development of a disease, for example preventing development of osteoarthritis in a subject having cartilage dysfunction.

Serving: A serving is the amount of food or beverage a person or animal would customarily eat in one time. The serving size can often times be found on the Nutrition Facts label on the beverage. Serving sizes are also shown on the USDA Food Pyramid. For beverages, such as juice or soda, a serving is usually represented in common household terms, such as cup or fluid ounce.

Shellfish: A term for mollusks and crustaceans used as food. Exemplary shellfish include clams, snails, mussels, oysters, scallops, shrimp, lobster, and crayfish. Components of the shell or exoskeleton of these organisms can be converted into GLCN using known techniques.

Shellfish protein: A protein present in a shellfish, such as those that are allergenic in humans having shellfish allergies. Exemplary shellfish proteins include, but are not limited to, shellfish muscle proteins, such as tropomyosin.

Skin disorder: A disease or disorder in a subject that negatively affects the skin, and benefits from collagen formation. Examples include, but are not limited to: a wound, wrinkles, and acne. When GLCN is used to treat a skin disorder, GLCN can be introduced into products used on the skin, such as topical lotions and creams. Alternatively or in addition, GLCN can be introduced into beverages and consumed by a subject in need of treatment or prevention of a skin disorder.

Subject: Living multicellular vertebrate organisms, a category which includes both human and veterinary subjects, for example, mammals, rodents, and birds.

Therapeutically Effective Amount: An amount sufficient to achieve a desired biological effect. In one example, it is an amount that is effective to alleviate or reduce symptoms associated with cartilage dysfunction, such as pain, swelling, and decreased mobility, by more than a desired amount. In another example, it is an amount that is effective to stabilize symptoms associated with cartilage dysfunction, such that the symptoms do not worsen. In particular examples, it is a concentration of GLCN (alone or in combination with other agents) that is effective to alleviate, reduce, or stabilize symptoms associated with cartilage dysfunction, such as in a subject to whom a GLCN-supplemented liquid, such as a beverage, is administered.

In one example, it is an amount that is effective to alleviate or reduce symptoms associated with a skin disorder, such as promoting the healing of a wound or reducing the appearance of wrinkles, by more than a desired amount. In another example, it is an amount that is effective to stabilize symptoms associated with a skin disorder, such that the symptoms do not worsen. In particular examples, it is a concentration of GLCN (alone or in combination with other agents) that is effective to alleviate, reduce, or stabilize symptoms associated with a skin disorder, such as in a subject to whom GLCN is administered.

In one example, a therapeutically effective amount also includes a quantity of GLCN sufficient to achieve a desired effect in a subject being treated. For instance, it can be an amount necessary to improve signs or symptoms a disease, such as osteoarthritis, a skin disorder, or a wound.

The GLCN-containing beverages disclosed herein have equal application in medical and veterinary settings. Therefore, the general term “subject being treated” is understood to include all animals (such as humans, apes, dogs, cats, horses, and cows) that require treatment of a cartilage dysfunction or skin disorder, such as a wound.

Thermal tolerance: Refers to the ability of GLCN to be exposed to a high temperature, such as those used in heat pasteurization, without a resulting significant adverse effect on the taste, color, odor, or texture of a GLCN liquid, such as a beverage, when GLCN is present in the liquid during pasteurization.

In particular examples, the amount of GLCN present in a GLCN beverage following exposure to a high temperature, demonstrating that GLCN is thermally tolerant, is at least 80%-100%, for example at least 90% -100%, for example at least 93%-100%, for example at least 95%-100%, for example at least 98%-100%, for example at least 90%-98%, or for example at least 93-98%. In other examples, the amount of GLCN present in a GLCN beverage following pasteurization is at least 80% of the amount of GLCN present prior to pasteurization, for example at least 85%, at least 90%, at least 92%, at least 93%, at least 94%, at least 95%, at least 98%, at least 99%, or even 100% (no loss of GLCN).

Treat: To alleviate or reduce one or more of the symptoms of a disorder, such as a cartilage dysfunction, wound or skin disorder, or to stabilize such a condition.

### Glucosamine-Supplemented Liquids

Disclosed are GLCN-supplemented liquids, such as beverages, which are exposed to high temperature conditions, such as those using during heat pasteurization of the liquid. The liquid supplemented with GLCN (such as a GLCN beverage) is exposed to one or more high temperatures, and the resulting taste, color, odor, and/or texture of the GLCN liquid is not significantly adversely affected. The results disclosed herein demonstrate that GLCN is heat tolerant and can be placed in liquids, such as beverages, prior to heating to high temperatures, such as those used during heat pasteurization. This is surprising given the current understanding of one of ordinary skill in the art that GLCN is inactivated at high temperatures.

The amount of GLCN added to a liquid, such as a beverage, can depend on the desired concentration of GLCN to be acheived. In certain examples, GLCN is present in the disclosed liquids in amounts effective for promoting the development of connective tissue in the body, alone or in combination with other agents, such as cartilage promoting agents. In some embodiments, daily GLCN dosages include at least about 250 mg, at least about 500 mg, at least about 1000 mg, at least about 2000 mg, or even about at least 3000 mg. Particular GLCN dosage ranges include, but are not limited to, a range of about 500 mg to about 3000 mg, such as about 1000 mg to about 2000 mg, such as about 1500 mg of GLCN.

Certain embodiments of the disclosed amounts of GLCN that can be included in a liquid, such as a beverage, include, about 0.001 g to about 20 g GLCN/serving, such as about 0.1 g to about 10 g GLCN/serving, or about 0.5 g to about 0.75 g GLCN/serving. Other examples include at least 0.01 g GLCN/serving, such as at least about 0.05 g GLCN/serving, at least about 0.1 g GLCN/serving, at least about 0.25 g GLCN/serving, at least about 0.5 g GLCN/serving, at least about 0.75 g GLCN/serving, at least about 1.0 g GLCN/serving, at least about 1.5 g GLCN/serving, at least about 3.0 g/serving, at least about 5.0 g GLCN/serving, at least about 10.0 g GLCN/serving, or even at least about 20.0 g GLCN/serving. In other examples, the amount of GLCN added is about 1 g GLCN/1000 g of product to about 1 g GLCN/0.1 g of product, such as about 1 g GLCN/10 g product to about 1 g GLCN/0.5 g product.

In certain embodiments, the GLCN liquids disclosed herein, such as beverages, are supplemented with one or more other cartilage supplements, vitamins, minerals, fats, proteins, carbohydrates, sweeteners, organic acids, glucose, unreacted chitin, and glucan conversion materials, such as melanoidins and levulinic acid, or combinations thereof In addition, other agents that treat cartilage dysfunction or skin disorders can also be included in the disclosed GLCN-supplemented liquids.

Melanoidins are relatively complex, high molecular weight, irregular polymers and are present in particular embodiments of the GLCN liquids. For example, particular embodiments of the disclosed GLCN liquids include from 0.001 to 15 wt. % melanoidins, or from 0.001 to 1.0 wt. % melanoidins or from 0.01 to 0.1 wt. % melanoidins. Without being tied to any particular theory, melanoidins are likely formed by the conversion of glucans to dextrose to hydroxymethylurfural AHMF) to produce the melanoidins. (The reaction may produce other glucan-derived products and amines from proteins in a biomass source as well as lipids in such a source.) Such a chemical process is the Maillard Reaction.

Levulinic acid (also known as acetyl-propionic acid) is present in particular embodiments of the disclosed GLCN liquids. Without being tied to any particular theory, levulinic acid is likely formed when glucans in the fungal biomass are converted to dextrose, which is converted to HMP to finally form formic and levulinic acids. Levulinic acid is a non-hazardous component that is a valuable acidulant used in such products as carbonated and fruit juice beverages, jams, and jellies. Thus, addition of embodiments of the GLCN compositions to such products provides an acidulant benefit as well as the benefits provided by the GLCN in the composition. Particular embodiments of the GLCN liquids include from 0.0001 to 1 wt. % levulinic acid, or from 0.001 to 0.7 wt. % levulinic acid or from 0.01 to 0.4 wt. % levulinic acid.

Because the melanoidins and levulinic acid are formed when producing the GLCN compositions according to the disclosed methods, no additional steps must be taken to include such components in the compositions. Melanoidins and levulinic acid were not expected in GLCN compositions derived from shellfish, and analysis of six lots of GLCN derived from shellfish (obtained from five different suppliers) did not contain any detectable amounts of melanoidins or levulinic acid.

With reference to Table 1, embodiments of the GLCN compositions useful for malting embodiments of the presently disclosed GLCN liquids, such as beverages, include GLCN derived from fungal biomass and can also include one or more of the listed components in Table 1, those shown in Table 2 and other components as discussed herein. Concentrations of each component can be within the ranges shown or varied by altering any of a variety of production parameters.

TABLE 1
Components that can be present in a GLCN liquid

| | Representative | Representative | Representative |
|---|---|---|---|
| Glucosamine | Embodiment | Embodiment | Embodiment |
| Composition | Percent | Percent | Percent |
| Components | by Weight | by Weight | by Weight |
| Glucosamine | 85-99.8 | 95-99.8 | 98-99.8 |
| Melanoidins | 0.001-15 | 0.001-1.0 | 0.01-0.1 |
| Levulinic Acid | 0.0001-1 | 0.001-0.7 | 0.01-0.4 |
| Dextrose | 0.001-10 | 0.001-5 | 0.001-2 |
| Citric Acid | 0.001-10 | 0.01-1.0 | 0.025-0.5 |

Two specific embodiments of the GLCN compositions are set forth in Table 2.

TABLE 2
Specific embodiments of GLCN liquids

| Composition | *Embodiment 1 | *Embodiment 2 |
|---|---|---|
| Component | (GP-11) | (GP-17C) |
| Ash Content | 0.03% | 0.02% |
| Si | 140 ppm | 150 ppm |
| Na | 10-100 ppm | 10-100 ppm |
| K | 10-100 ppm | 10-100 ppm |
| Ca | 10-100 ppm | 10-100 ppm |
| HCL | 0.16% | 0.19% |
| Citric Acid | 0.045% | 0.074% |
| Levulinic Acid | 0.39% | 0.3% |
| Melanoidins | 0.04-0.07% | 0.02-0.03% |
| Water-insoluble matter soluble in | 0.05% | 0.02% |
| gastric juice at ˜40° | | |

*Percentages listed are percents by weight

Also disclosed are methods for producing GLCN compositions useful in forming embodiments of the presently disclosed GLCN liquids from fungal biomass sources, including producing such compositions by acid hydrolysis of fungal biomass. Acid hydrolysis breaks ether linkages in the biomass and deacetylates chitin molecules to generate free GLCN. Acid hydrolysis can break the chitin into GLCN, but leaves the GLCN molecule substantially intact. Depending upon the acid hydrolysis parameters, acid hydrolysis conditions break down other components (such as glucans, proteins, and lipids) that exist in the fungal biomass.

In one specific embodiment of the disclosed method for producing GLCN compositions from fungal biomass, acid hydrolysis is performed by treating fungal biomass for a relatively long period of time, for example greater than 4 hours, in a relatively aggressive acid solution.

Chitin-containing fungal biomass may first be reacted in a relatively aggressive acidic solution. Relatively strong (aggressive) acids can be used to hydrolyze the fungal biomass, including acids of concentrations less than 50 percent. Acids of concentrations of from about 5 to about 25 percent are also suitable. Suitable strong acids include hydrochloric, sulfuric, phosphoric, and citric acid at appropriate concentrations.

The aggressive acid treatment mixture containing the biomass, acid, and water is heated and maintained at a relatively elevated temperature. The mixture is usually heated to a temperature at or near its boiling point (typically 90° C. to 106° C.) and maintained under reflux conditions for 5 hours or greater, more typically greater than 8 hours, and usually less than 16 hours. The reaction may continue long enough to have a complete breakdown of the chitin, but not so long as to be inefficient or to excessively decompose the GLCN compositions.

Although reaction in the relatively aggressive acid solution produces a GLCN composition, subsequent purification steps can be taken. A first purification step may include a separation step, such as filtration, to remove particulate impurities, resulting in a substantially clear solution of the GLCN composition. The solution contains an embodiment of GLCN composition as well as small quantities of glucose and other components of the composition. The GLCN composition can be concentrated and some of the acid recovered can be recycled and reused.

In yet other examples, GLCN is a GLCN composition that is derived from animal cartilage (for example see U.S. Pat. No. 5,922,692). Suitable starting materials include vertebrate connective tissue, such as from a cow, pig, or chicken. Briefly, to prepare GLCN from cartilage, raw vertebrate connective tissue is disintegrated into an aggregation of particles having a substantially homogenous particle size, such as by emulsification, thereby forming liquefied connective tissue. The liquefied connective tissue is then thermally processed to generate a product rich in GLCN.

In particular examples, GLCN is a GLCN composition derived from bacteria (for example see U.S. Pat. No. 6,372,457). For example, GLCN can be produced by fermentation of a microorganism. Briefly, a microorganism having a genetic modification in an amino sugar metabolic pathway is cultured in a fermentation medium. GLCN can then be recovered from the fermentation medium. Exemplary amino sugar metabolic pathways include a pathway for transport of glucosamine out of the microorganism or a pathway for transport of glucosamine into the microorganism.

In one embodiment, liquids (such as beverages) suitable for use in the present disclosure include those pasteurized after GLCN is included in the liquid. For example, GLCN-supplemented liquids can be prepared using heat pasteurization at a high temperature, wherein GLCN is present in the liquid during the heat pasteurization. In certain examples, pasteurization is used to kill a substantial amount of undesirable bacteria.

GLCN-supplemented liquids, such as beverages, can be heat pasteurized at a temperature that will destroy a substantial amount of undesirable organisms, such as bacteria. Particular non-limiting heat pasteurization temperatures include, at least about 160° F., at least about 180° F., at least about 200° F., at least about 220° F., at least about 280° F., and even such as at least about 300° F. Particular examples of a pasteurization temperature are about 160° F., about 194° F., about 220° F., or even about 280° F. In one example, liquids supplemented with GLCN are exposed to both high temperature and pressure. One example of pasteurization conditions is about 121° C. at 1 atm for 15 minutes.

In a particular example, GLCN-supplemented liquids are heated to at least about 170° F. For example, GLCN can be added to coffee, tea, or cocoa. In one example, GLCN is included in a coffee, tea, or cocoa mixture (such as a pre-prepared packet) to which boiling or heated water (or other liquid such as milk) is added.

Liquids such as beverages supplemented with GLCN, such as those including at least about 0.01 g GLCN per serving, and at least about 0.001 wt. % levulinic acid are encompassed by this disclosure. In particular embodiments, such GLCN-supplemented liquids are at a temperature of at least 160° F. In specific examples, a GLCN liquid contains no detectable shellfish proteins, such as allergenic shellfish proteins (those causing an allergic reaction in some humans).

### Methods of Preparing Glucosamine-Supplemented Liquids

Methods of preparing liquids that include GLCN are disclosed. In one example, the method includes heat pasteurizing the liquid, such as a beverage, to a high temperature wherein GLCN is present in the liquid during the exposure to a high temperature. In another example, the method includes combining GLCN and a liquid, such as a beverage, thereby forming a GLCN-liquid, then heat pasteurizing the GLCN-liquid at a high temperature.

In certain embodiments, the amount of GLCN present in the GLCN-liquid prior to the heat pasteurization is substantially similar to an amount of GLCN in the GLCN-liquid after heat pasteurizing. In one example, the amount of GLCN in the GLCN liquid after heat-pasteurizing is at least about 80% of the amount of GLCN in the GLCN liquid prior to heat-pasteurizing, such as at least about 90%, at least about 95%, at least about 98%, or even about at least 100%.

### Treatment Using Glucosamine

A method of treating a cartilage dysfunction in a subject by administering the disclosed GLCN liquids is disclosed. In some examples, treatment alleviates or reduces the symptoms of cartilage dysfunction, such as increases joint mobility, reduces pain, or reduces swelling in the subject. In certain embodiments, treatment stabilizes the symptoms of cartilage dysfunction, such that the cartilage dysfunction is not exacerbated. Examples of cartilage dysfunction include, but are not limited to, joint pain and osteoarthritis.

Also disclosed are methods of treating a skin disorder in a subject by administering the disclosed GLCN liquids, such as beverages, to a subject. In some examples, treatment alleviates or reduces the symptoms of a skin disorder, such as promotes wound healing in the subject. In some examples, treatment stabilizes the symptoms of a skin disorder, such that the skin disorder is not exacerbated. Examples of skin disorders include, but are not limited to, wounds and wrinkles.

A method for treating food allergies in a subject by administering the disclosed GLCN liquids to the subject is disclosed. In some examples, treatment alleviates or reduces the symptoms of a food allergy, such as reduces the inflammatory response to the food in the subject. In some examples, treatment stabilizes the symptoms of a food allergy, such that the food allergy is not exacerbated.

The subject treated can be a human or veterinary subject suffering from cartilage dysfunction, skin disorder or food allergy (for example see WO 93/14766A1). An effective amount of GLCN can be administered in a single serving, or in several servings, for example daily, during a course of treatment. However, the effective amount can be dependent on the subject being treated, the severity and type of the condition being treated, and the manner of administration.

A typical amount of GLCN delivered in dietary supplement products is about 1500 mg per day, in a single or in multiple administrations. For example, if the subject was to receive multiple administrations in a single day, the subject might receive three servings of GLCN, each containing about 500 mg GLCN. GLCN can be administered at about at least about 0.001 g per day. In one example, GLCN is administered at about 750 mg GLCN per day. In other examples, GLCN is administered at about at least 10 mg per day, such as about at least 50 mg per day, about at least 100 mg per day, about at least 250 mg per day, about at least 500 mg per day, about at least 750 mg per day, about at least 1.0 g per day, about at least 1.5 g per day, about at least 3.0 g per day, about at least 5.0 g per day, about at least 10.0 g per day, or even about at least 20.0 g per day.

### EXAMPLE 1

### Allsport® Sports Drink

Allsport® sports drink (The Monarch Company, Atlanta, Ga.) was used as the basis for incorporation of samples. Citrus Slam flavor was chosen because of its light color, which allows panelists observe color change. According to the manufacturer, a 20-ounce bottle of Allsport® contains 2.5 servings.

For servings that included GLCN, the serving had 0.75 g GLCN added, which reflects a typical amount of GLCN delivered in dietary supplement products. Therefore, 1.875 g (0.75 g/serving GLCN×2.5 servings/bottle=1.875 g GLCN/bottle) of GLCN was added to a 20-ounce bottle of Allsport®. After the bottles were prepared, the contents were heated to 195±4° F. for 42±4 seconds to simulate a heat pasteurization step. Samples were heated in a 1000 watt Amana Radarange microwave oven in foam cups for the same amount of time. For a room temperature beverage, 150 seconds of heating time was necessary, and for a refrigerated beverage, 180 seconds was required. The beverages were cooled in their original bottles, ¾ submerged in an icewater bath, and hand rotated to simulate flash cooling.

Samples were tested within one day of preparing. Samples were tested using a “Difference From Control” test, using a marked control, and a blind control was included. Panelists were asked to compare each sample to the control and comment. Panelists received the following instructions:
1. Taste the control sample first.
2. Compare all other samples to the control and write descriptors in the table.

3. Numerically rate whether the samples are better or worse than the control, using this scale:

| | | | | | | | | | | |
|---|---|---|---|---|---|---|---|---|---|---|
| −5 | −4 | −3 | −2 | −1 | 0 | +1 | +2 | +3 | +4 | +5 |

| Worse | Same as | Better |
|---|---|---|
| than | control | than |
| control | | control |

4. Please do not discuss the results with any other panelists until all sheets are turned in.

The 12 Panelists' results are shown in Table 3.

TABLE 3
Results of Allsport ® sports drink.*

| | | | Mouthfeel/ | | |
|---|---|---|---|---|---|
| Sample | Color | Odor | texture | Taste | Comments (other than “same”) |
| Control | +0.1 | 0 | +0.4 | −0.1 | Less sour than others, sweeter (2), more of a |
| | | | | | carbonated feel, stronger odor (2), more |
| | | | | | tangerine taste, mouthfeel thicker |
| Fungal | 0 | −0.3 | +0.1 | −0.3 | After/off-taste (2), “vitamin” like taste, |
| GLCN | | | | | sweeter, less odor, sour, yucky |
| Shellfish | −0.1 | −0.3 | −0.2 | −0.7 | Sweeter (2), sour, crystals on top, less odor, |
| GLCN | | | | | mouthfeel thicker, less tangy, flat and yucky |

*Results are the average rounded to nearest tenth.

As shown in Table 3, it was difficult for the panelists to ascertain differences between the samples. The control was not clearly differentiated from GLCN. These findings demonstrate that taste and other factors are not adversely affected when GLCN is used in a high temperature beverage application, such as those that require heat pasteurization. That is, the presence of GLCN did not significantly affect the taste of the final GLCN beverage, even though GLCN was exposed to a high temperature.

### EXAMPLE 2

### Libby's White Grape Juicy Juice®

Libby's White Grape Juicy Juice® was used as the basis for incorporation of samples. White Grape was chosen due to its light color, to help panelists observe any color change. According to the manufacturer, a 64 ounce bottle contains 8 servings of 8 ounces each.

For servings that included GLCN, the serving (8 ounces) had 0.75 g GLCN added, which reflects a typical amount of GLCN delivered in dietary supplement products. 16-ounce portions of juice were used. To the appropriate batches, 1.5 g of GLCN (16 ounces is two servings, and 2 servings×0.75 g/serving=1.5 g) was added. After preparing the samples, they were heated to 195∓4° F. in a microwave, held at that temperature for 42 seconds to simulate heat pasteurization, then cooled as described in Example 1.

Samples were tested according to the methodology and instructions in Example 1. The 12 Panelists' results are shown in Table 4.

TABLE 4
Libby's White Grape Juicy Juice ® results.*

| | | | Mouthfeel/ | | |
|---|---|---|---|---|---|
| Sample | Color | Odor | texture | Taste | Comments (other than “same”) |
| Control | +0.1 | 0 | 0 | 0 | Slight off taste, more lingering taste |
| Fungal | −0.2 | −0.3 | −0.3 | −0.6 | Darker (2), thicker mouthfeel (2), sweeter (2), |
| GLCN | | | | | stale/weaker (2), smooth, soapy, lasting |
| | | | | | aftertaste, tart, odor masked |
| Shellfish | −0.4 | −0.1 | −0.3 | −1.1 | Darker (3), thicker mouthfeel, sweeter (2), |
| GLCN | | | | | soapy (2), tart, bitter, less sweet, less fruity (2) |

*Results are the average rounded to nearest tenth.

The data shown in Table 4 is validated by identification of the control, based on the control score of near “0” in all categories. As shown in Table 4, it was difficult for the panelists to ascertain differences between the samples. These results further demonstrate that taste and other factors are not adversely affected when GLCN is used in a high temperature beverage application, such as those including a heat pasteurization step.

### EXAMPLE 3

### Hy-Vee® Healthy Recipe Tomato Soup

Hy-Vee® Healthy Recipe Tomato Soup was used as the basis for incorporation of samples. Tomato soup was chosen for its even texture, and “healthy” aspect when compared to other soups. According to the manufacturer, one can makes 2.5 servings.

For servings that included GLCN, the serving had 0.75 g GLCN added, which reflects a typical amount of GLCN delivered in dietary supplement products. Each batch prepared 2.5 servings. For batches 2-4, 1.875 g GLCN was added (0.75 g GLCN/serving×2.5 servings=1.875 g GLCN/batch). The recipe on the can was used. The soup can contents were poured into a microwaveable container, one can of water slowly stirred in, and GLCN added (where applicable) and the mixture stirred well. The container was covered with vented plastic wrap (turn back edge of wrap to form small opening for steam to escape), and the mixture heated in a 1000 watt Amana Radarange at high power for 2 minutes. The sample was not allowed to come to a boil.

Samples were tested according to the methodology and instructions in Example 1. The 8 Panelists' results are shown in Table 5.

TABLE 5
Hy-Vee ® Healthy Recipe Tomato Soup results.*

| | | | Mouthfeel/ | | |
|---|---|---|---|---|---|
| Sample | Color | Odor | texture | Taste | Comments (other than “same”) |
| Control | −0.2 | 0 | −0.1 | +0.2 | less tangy (2), lighter in color |
| Fungal | 0 | 0 | −0.1 | −0.3 | Off flavor, a little metallic, flat/sour taste but |
| GLCN | | | | | barely perceptible, more tangy w/ acidic |
| | | | | | aftertaste (like black pepper bad been added), |
| | | | | | milder, maybe sweeter, less tangy aftertaste |
| Shellfish | −0.2 | 0 | −0.3 | −0.6 | Slight off flavor, dirty odor, flat/sour taste but |
| GLCN | | | | | barely perceptible, lighter in color, off |
| | | | | | aftertaste, bitter, chalky in texture, sour |

*Results are the average rounded to nearest tenth.

The results shown in Table 5 are validated by identification of the control; based on the control score of near “0” in all categories. It was difficult for the panelists to ascertain differences between the samples. These results demonstrate that taste and other factors are not adversely affected when GLCN is used in a high temperature food application, such as those that include heating or boiling prior to consumption.

### EXAMPLE 4

### Lemonade

Lemonade made from scratch was used as the basis for incorporation of samples. To prepare the lemonade, the following recipe was used: 10.8 g dry dextrose, 82.0 g 42 high fructose corn syrup (HFCS), 5.6 g citric acid, 0.5 g sodium citrate, 5.7 g GLCN or NAG, then add water to one quart. Lemonade was prepared for sensory testing as follows:
Batch 1. Control Lemonade (no fungal glucosamine)
Batch 2. Lemonade+fungal glucosamine (pH 3-4)
Batch 3. Lemonade+fungal glucosamine (pH 3-4) heated to 71° C. for 20 minutes
Batch 4. Lemonade+fungal glucosamine (pH 3-4) heated to 90° C. for 20 seconds, flash cooled in ice water bath
Batch 5. Lemonade+fungal glucosamine (pH 3-4) boiled on stove for 5 minutes, flash cooled in ice water bath

The recipe prepared one quart, so each batch consisted of 32 ounces, or four 8-ounce servings. For servings that included GLCN, the serving (8 ounces) had 1.425 g GLCN (1.425 g/serving×4 servings/batch=5.7 g/batch), which reflects a typical amount of GLCN delivered in dietary supplement products.

Samples were tested according to the methodology and instructions in Example 1. The four Panelists' results are shown in Table 6.

TABLE 6
Results of Lemonade.*

| | Actual | | 30 sec | | | Sensory Panel |
|---|---|---|---|---|---|---|
| Sample | Temp ° C. | Initial pH | Temp ° C. | Mouthfeel | Taste | Observations |
| Control | Not heated | 3.13 | N/A | 0.5 | 0 | Tart, sweet, bitter |
| | | | | | | (2), astringent |
| GLCN not | Not heated | 3.10 | N/A | 0.8 | 2.8 | More mild, more |
| heated | | | | | | sour than sweet, |
| | | | | | | sweeter (2) |
| GLCN | 71 | 3.03 | 67 | 1.3 | 1.5 | Stronger sweet (2), |
| 71° C. | | | | | | tart, less sweet, more |
| | | | | | | mild |
| GLCN | 90 | 3.05 | 74 | 1.3 | 1.8 | Chemical taste, sweet |
| 90° C. | | | | | | (2), flat taste (2), |
| | | | | | | strong taste |
| GLCN | 97 | 3.30 | 73 | 0.8 | −1.0 | Less sweet, |
| boiled | | | | | | cardboard essence, |
| | | | | | | off taste, less flavor, |
| | | | | | | bitter |

*Values shown are the average rounded to nearest tenth.

As shown in Table 6, these results further demonstrate that taste and mouthfeel are not adversely affected when GLCN is used in a high temperature food application, such as those that include heating or even boiling prior to consumption. In several samples, the presence of GLCN improved taste and mouthfeel relative to the control samples.

### EXAMPLE 5

### Determination of the Amount of GLCN Present Following Heating

To determine the amount of GLCN that remains following exposing GLCN beverages to a high temperature, such as those used in pasteurization, the following methods were used.

A beverage sample as described in the preceding examples containing S to 20 mg of GLCN was dispersed in 25 g of 1.0 N HCl in a 50-mL polypropylene centrifuge tube and capped tightly. The sample was mixed for 30 seconds using a vortex mixer, then placed in a water bath at 37° C. The sample was removed from the water bath at 15-minute intervals, mixed for 30 seconds on a vortex mixer, then returned to the water bath. This cycle was repeated until the sample had been in the water bath for one hour.

After heating, the sample was mixed for 30 seconds on a vortex mixer, then centrifuged for 10 minutes to separate the liquid and solid phases. Fats, oils or lipids in the sample formed a third layer at the top of the tube. A 1-g aliquot of the aqueous sample portion was diluted 100-fold with deionized water, then transferred to an autosampler vial with filter cap.

The free glucosamine in prepared samples was determined using high performance anion-exchange chromatography with pulsed amperometric detection (IPAEC-PAD). The system included an EG40 eluent generator, GP50 gradient pump, AS40 autosampler, LC25 column oven, and ED40 electrochemical detector, all produced by Dionex Corporation (Sunnyvale, Calif.).

The method was adapted from Dionex Corporation Technical Note 40. A Dionex CarboPac PA-20 column was used in place of the PA-10 described in the Technical Note. The eluent was 8 mM KOH at 0.5 mL/min. The column and detector were maintained at 30° C. The injection volume was 10 μL. The standard was glucosamine hydrochloride at 10.8 mg/L. Fermentation broth samples were diluted five-fold with deionized water, ASTM Type II, and filtered through 0.2 μ vial filters in the autosampler. Multiple standards were analyzed before and after each sample set. The results are shown in Table 7.

TABLE 7
Percent of GLCN recovery following heating.

| Sample | Temp ° C. | Time | % GlcNHCl Recovered |
|---|---|---|---|
| Lemonade | Room temperature | | 102 |
| Lemonade | 90 | 20 sec | 101 |
| Lemonade | 71 | 20 min | 101 |
| Lemonade | boil | 5 min | 100 |
| Grape juice | boil | 5 min | 102 |

As shown in Table 7, there was little degradation of GLCN when the beverages were boiled. The amount of recovery for GLCN was about 100%. Therefore, in contrast to the previous understanding of those skilled in the art, GLCN is not substantially degraded or inactivated following exposures to high temperatures, such as those used in heat pasteurization.

In view of the many possible embodiments to which the principles of this disclosure may be applied, it should be recognized that the illustrated embodiments are only particular examples of the disclosure and should not be taken as a limitation on the scope of the disclosure. Rather, the scope of the disclosure is in accord with the following claims. We therefore claim all that comes within the scope and spirit of these claims.

1. A method for making a beverage comprising:
providing a beverage;
providing an amount of glucosamine (GLCN);
mixing the beverage and the GLCN, thereby forming a GLCN beverage; and
heat-pasteurizing the GLCN beverage at a high temperature for a time sufficient to reduce colony forming units (cfu) by at least about 50%.

2. The method of claim 1, wherein heat-pasteurizing the GLCN beverage comprises heating the GLCN beverage to at least about 160° F.

3. The method of claim 1, wherein heat-pasteurizing the GLCN beverage comprises heating the GLCN beverage to at least about 200° F.

4. The method of claim 1, wherein heat-pasteurizing the GLCN beverage comprises heating the GLCN beverage to a temperature in a range of from about 160° F. to about 300° F.:

5. The method of claim 1, wherein the GLCN beverage is heat-pasteurized for a time period from about 1 second to about 5 minutes.

6. The method of claim 1, wherein the amount of GLCN added to the beverage is at least about 0.1 g GLCN per serving

7. The method of claim 6, wherein the amount of GLCN added to the beverage is at least about 0.25 g GLCN per serving.

8. A method for making a beverage comprising:
providing a beverage;
providing a first amount of GLCN;
mixing the beverage and the GLCN, thereby forming a GLCN beverage; and
heat-pasteurizing the GLCN-beverage, wherein the amount of GLCN in the GLCN beverage prior to heat-pasteurizing is substantially similar to a second amount of GLCN in the GLCN beverage after heat-pasteurizing.

9. The method of claim 8, wherein the second amount of GLCN in the GLCN beverage after heat-pasteurizing is at least about 80% of the first amount of GLCN in the GLCN beverage prior to heat-pasteurizing.

10. The method of claim 1, wherein the GLCN is derived from a fungal biomass containing chitin.

11. A beverage made by the method claim 1.

12. A beverage comprising:
at least about 0.01 g per serving of GLCN; and
at least about 0.0001 wt. % levulinic acid.

13. A beverage comprising:
at least about 0.01 g per serving of GLCN; and
at least about 0.0001 wt. % melanoidins.

14. The beverage of claim 13, wherein the beverage does not contain shellfish proteins.

15. A beverage comprising:
at least about 0.01 g per serving of GLCN;
at least about 0.0001 wt. % levulinic acid and/or at least about 0.0001 wt. % melanoidins; and
wherein the beverage is at least about 160° F.

16. The beverage of claim 13, wherein the beverage comprises at least about 0.25 g GLCN per serving.

17. The beverage of claim 15, wherein the beverage comprises at least about 0.25 g GLCN per serving.
